(19) United States
(12) Patent Application Publication
(10) Pub. No.: US 2009/0036518 A1
(43) Pub. Date: Feb. 5, 2009

(54) PHARMACEUTICAL COMPOSITION CONTAINING FLAVONOIDS

(75) Inventor: Wun-Chang Ko, Taipei (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: TAIPEI MEDICAL UNIVERSITY, Taipei (TW)

(21) Appl. No.: 12/241,822

(22) Filed: Sep. 30, 2008

Related U.S. Application Data
(63) Continuation-in-part of application No. 11/397,203, filed on Apr. 4, 2006.

(30) Foreign Application Priority Data
Apr. 12, 2005 (TW) 094111580

(51) Int. Cl. A61K 31/353; A61P 11/06; A61P 11/00

(52) U.S. Cl. 514/456

(57) ABSTRACT

A pharmaceutical composition is provided, where the pharmaceutical composition contains formula (I), (II), or (III) flavonoids which possess (PDE4), as a main ingredient. Especially, this composition has a binding affinity of high affinity rolipram binding sites (HARBS) of a PDE4 lower than a binding affinity of low affinity rolipram binding sites (LARBS) of the PDE4 is used in the treatment of asthma, chronic obstructive pulmonary disease (COPD), or chronic inflammation, and has bronchodilatory effects. In addition, whether the above-mentioned flavonoids have side effects, such as nausea, vomiting, gastric hypersecretion, etc., in accordance with their binding affinity to HARBS of particulates of brain cells are disclosed.

### FIELD OF THE INVENTION

The present invention is a CIP application of the parent application “Pharmaceutical Composition Containing Flavonoids” bearing on the serial no. and filed on Apr. 4, 2006. The present invention relates to a medical composition, and more particularly to a composition including the flavonoid compound, which possesses inhibition on the phosphodiesterase (PDE)4 and has a binding affinity of high affinity rolipram binding sites (HARBS) of the PDE4 lower than a binding affinity of low affinity rolipram binding sites (LARBS) of the PDE4.

### BACKGROUND OF THE INVENTION

PDEs have been classified according to their primary protein and cDNA sequences, co-factor and substrate specificities, and pharmacological roles. Giembycz has disclosed that the PDEs is classified to at least 11 distinct enzyme families that hydrolyze cAMP and/or cGMP [1]. The PDE1-5 isozymes are characterized as being calcium/calmodulin-dependent (PDE1), cGMP-stimulated (PDE2), cGMP inhibited (PDE3), cAMP-specific (PDE4), and cGMP-specific (PDE5) respectively. The PDE1-5 isozymes have been found to be present in canine trachea [2], human bronchi [3], and guinea pig lung [4]. In the guinea pig airway, the PDE3 and PDE4 have been identified, but other isozymes might also be present [5].

It is known that the adenylyl cyclase and the PDEs are mature enzymes responsible for modulating the level of the cytosolic signal transduction material, the cAMP, where the adenylyl cyclase is responsible for the production of the biologically active cAMP from the substrate ATP, and the PDEs are responsible for degrading the biologically active cAMP to the biological inactive molecule 5′-AMP.

The atopic asthma is a chronic inflammatory disorder of the airways. Busse and Lemanske [6], and Maddox and Schwartz [7] have disclosed that atopic asthma is characterized by reversible and recurrent of acute bronchial obstruction and airway hyperreactivity (AHR) with the following mechanism. Once inhaled the antigen in the airway, the antigen would bind to T cells and these cells in turn induce the production of cytokines, such as interleukin (IL)-4, IL-13, and IL-5. IL-4 and IL-13 will subsequently bind with B cells for producing immunoglobulin E (IgE) antibodies. Once synthesized and released by B cells, IgE antibodies briefly circulate in the blood before binding to IgE-bound high-affinity Fc receptors (FcεRI) on the surface of mast cells. And exposing to the allergen once again, the antigen will cross-link with the mast-cell-bound IgE. This cross-linking induces the mast cell to release the chemical cytokines and cysteinyl-leukotrienes that lead to the initiation of the inflammatory reaction of the eosinophils. In addition, Willis-Karp [8] has disclosed that through the release of cytokines IL-5 from T cells, it directly leads to the production of the eosinophils, where the production of the eosinophils in the airway is highly related to the atopic airway inflammatory response, and the production of the eosinophils in the lung erupts with the remodeling of the airway and change the airway tone controlled by the nervous system. Moreover, Kumar [9] has disclosed that the shedding of the epithelium causes the acute bronchial obstruction and airway hyperreactivity (AHR).

Presently, besides the traditional drug for asthma, the aminophylline, the other spray selective agonists, the β-adrenoceptor, for emergency use also releases the bronchial obstruction. However, since frequently using the spray leads to the decrease of the number of the receptors (down-regulation), the curative effect of the β-adrenoceptor will decrease in accordance therewith. The steroids are used for the inflammatory response. However, there are side effects, such as development of moon-face, broader shoulders, adrenal gland atrophy, decrease in immunity, and so on when take steroids for a long time. Moreover, even the spray steroid taken by inhalation also has the problems of Candida albicans infections. Hence, people are looking for new drugs in the treatment of asthma.

Flavonoids are naturally polyphenolic compounds and are widely distributed in the plants and vegetables. It has been reported that the daily diary intake of flavonoids for westerner per day is 1 g. There are approximately 4000 kinds of the naturally polyphenolic compounds in the world, and based on the structures thereof, they are classified into four groups, flavones, flavonols, flavanones and isoflavones.

Flavonoids are reported to have anti-inflammatory and immuno-regulatory potentials. Baumann et al have disclosed the inhibition of flavonoids for cyclooxygenase in the cell [10]. Havsteen has disclosed that flavonoids inhibit lipoxygenase and have anti-inflammatory and antioxidant potentials [11]. Also, flavonoids are reported to have anticancer and anti-viral potentials [12], and to have potentials for being an angiogenic inhibitor [13] respectively. In addition, in RAW 264.7 cells, the inhibition of nitric oxide (NO) production and of inducible NO synthase (iNOS) expression by flavonoids has been reported by Kim et al [14]. Moreover, it has been reported that proteoglycan and/or the anti-histamines is administered with or without flavonoids for treating diseases induced by activation of mast cell (such as allergy), though flavonoids are not the principal component [15].

Akiyama et al have disclosed that genistein is a selective inhibitor of tyrosine-specific protein kinase [16]. However, besides being a selective inhibitor of tyrosine-specific protein kinase, genistein with its tyrosine kinase-independent inhibition of cyclic-AMP PDE has been reported by Nichols and Morimoto [17]. Nichols and Morimoto further investigated the inhibition by genistein on the PDE1, 3 and 4, and, reported that genistein is more selective to inhibit PDE4 with the IC₅₀ thereof being 5 μM [18], where no further investigation of the mode of inhibition therefor has been carried out.

Underwood et al have reported that all cyclic AMP-specific PDE4 inhibitors have inhibitory effect on the antigen-induced bronchoconstriction [19]. In addition, Underwood et al also reported that the combination usages of genistein with selective inhibitor of the PDE4 with the dual PDE¾ inhibitor effectively inhibit the bronchospasm and the pulmonary eosinophil influx both in vivo and in vitro [20].

Recently, in United States of American and Europe, the selective inhibitors for the PDE4 are considered as the important materials for treating asthma or chronic obstructive pulmonary disease (COPD). In addition, the clinical trials thereof are considered safe and effective, whereas they have side effects, such as vomiting, gastric hypersecretion, etc. [21]. Take the most typical and highly selective PDE4 inhibitor, such as rolipram, for example. In the brain, there are two binding sites for rolipram, which are high (HARBS) and low affinity rolipram binding sites (LARBS), while in the periphery of bronchi and lung there exist only LARBS. Generally, the anti-inflammatory and the bronchodilatory effects of rolipram are considered as its ability to bind with LARBS [21]. The binding ability of rolipram is similar to its ability of inhibition on PDE4 catalytic activity [22], and the side effects are correlated to its binding ability to HARBS [1]. The brain HARBS and peripheral LARBS are respectively called PDE4_{H} and PDE4_{L}, where an effective concentration of a compound at which a half maximal PDE4_{H} being displaced by the compound is called EC₅₀ and an inhibitory concentration of a compound at which a half maximal PDE4 activity being inhibited by the compound is called IC₅₀. In the present invention, the IC₅₀ values are reported to be similar to the effective concentration that a half maximal PDE4_{L} is displaced [22]. The EC₅₀ of rolipram for binding PDE4_{H} is about 2 nM, and the IC₅₀ of rolipram for binding PDE4_{L} is about 1 μM [23]. Accordingly, the ratio of the EC₅₀ to the IC₅₀, which will be called the PDE4_{H}/PDE4_{L} ratio in the following description, of rolipram is only 0.002, and hence the side effects of rolipram are too big to take it as a therapeutic drug. Therefore, the pharmaceutical factories in the world are trying to develop drugs with high PDE4_{H}/PDE4_{L} ratio for separating the side effects from the main therapeutic effects, while some progresses are obtained. For example, roflumilast has been in clinical trial phase-III for treating both asthma and COPD until 2005, and cilomilast in phase-II for treating asthma until 2003, and in phase-III for COPD, respectively, wherein the PDE4_{H}/PDE4_{L} ratio of roflumilast is 3[24, 25], and the PDE4_{H}/PDE4_{L} ratio of cilomilast is about 1 [25]. Although the respective PDE4_{H}/PDE4_{L} ratios of roflumilast and cilomilast are much higher than that of rolipram, they are not good enough. AWD-12-281, a newly developed compound with a much higher PDE4_{H}/PDE4_{L} ratio about 11 [26], enters the clinical trial phase-II. It seems having a good perspective.

In order to overcome the foresaid drawbacks, the present invention provides a medical composition including the flavonoid compound, which possesses selective inhibition on the PDE4 and has a binding affinity of HARBS of the PDE4 lower than a binding affinity of LARBS of the PDE4.

### SUMMARY OF THE INVENTION

In accordance with an aspect of the present invention, a medical composition including a flavonoid compound with the formula (I), (II) or (III), as an active constituent for inhibiting the PDE4 and having the high PDE4_{H}/PDE4_{L} ratio is provided.

In accordance with another aspect of the present invention, a medical composition for treating the asthma, the chronic obstructive pulmonary disease, or the chronic inflammation is provided, wherein the medical composition includes a flavonoid compound with the formula (I), (II) or (III) as an active constituent, and the flavonoid compound inhibits the PDE4 and having the high PDE4_{H}/PDE4_{L} ratio. The present invention further provides determining that whether the above-mentioned flavonoid compounds have side effects, such as vomiting, gastric hypersecretion, etc., is in accordance with whether the above-mentioned flavonoid compounds bind to the particulate HARBS of the brain cells.

The above objects and advantages of the present invention will become more readily apparent to those ordinarily skilled in the art after reviewing the detailed description of the preferred embodiments of the present invention and accompanying drawings.

The mentioned flavonoids with the formula (I), (II) or (III) of the present invention, preferably, hesperetin, quercetin and the derivatives thereof have high PDE4_{H}/PDE4_{L} ratios. Hence, mentioned flavonoids with little side effects are hopeful to be the effective drugs against asthma, COPD and inflammation (including airway inflammation, arthritis and rheumatoid arthritis).

The above objects and advantages of the present invention will become more readily apparent to those ordinarily skilled in the art after reviewing the following detailed description and accompanying drawings, in which:

### BRIEF DESCRIPTION OF THE DRAWINGS

FIG. 1A is a drawing showing the cAMP hydrolysis enzyme activity for PDE4 in relation to the treatment of hesperetin derivative of hesperetin-5,7,3′-O-trimethylether (HTME) at various concentrations;

FIG. 1B is a drawing showing the cAMP hydrolysis enzyme activity for PDE4 in relation to the treatment of hesperetin derivative of hesperetin-7,3′-O-dimethylether (HDME) at various concentrations;

FIG. 1C is a drawing showing the cAMP hydrolysis enzyme activity for PDE4 in relation to the treatment of rolipram at various concentrations;

FIG. 1D is a drawing showing the cAMP hydrolysis enzyme activity for PDE4 in relation to the treatment of Ro 20-1724 at various concentrations.

FIG. 2A is a drawing showing the replacements of [³H]-rolipram bound on HARBSs by rolipram and Ro 20-1724, and FIGS. 2B to 2H are drawings showing the replacements of [³H]-rolipram bound on HARBSs by quercetin, 3-MQ, ayanin, QTME, QPME, QPA and QMTA respectively.

### DETAILED DESCRIPTION OF THE PREFERRED EMBODIMENT

The invention is described more specifically with reference to the following embodiments. It is to be noted that the following descriptions of preferred embodiments of this invention are presented herein for the purposes of illustration and description only; it is not intended to be exhaustive or to be limited to the precise form disclosed.

Reagents and Drugs

Hesperetin, quercetin, other flavonoides listed in the following Table 1, Bis-tris base, Trizma base, D,L-dithiothreitol, benzamidine, EDTA, EGTA, PMSF, BSA, cyclic AMP, cyclic GMP, calmodulin, Dowex resin, DMSO, and Crotalus atrox snake venom, etc. are purchased from Sigma Chemical (St. Louis, Mo., USA). [³H]cAMP, [³H]cGMP, Q-Sepharose, and calmodulin-agarose are purchased from Amersham Pharmacia Biotech (Buchinghamshire, UK). Vinpocetin and Ro 20-1724 are purchased from Biomol (Plymouth Meeting, Pa., USA). Ethyleneglycol is purchased from Merck (KgaA, Darmstadt, Germany). Prunetin is purchased from Fluka Chemie (Gmbh CH-9471 Buchs, Switzerland). Other reagents, such as CaCl₂, MgCl₂, and NaCl, are of analytical grade.

The above-mentioned genistein, daidzein, biochanin A, prunetin and vinpocetin are dissolved in a mixture of DMSO and ethyl alcohol (1:1). Quercetin is dissolved in a mixture of ethyl alcohol and DMSO (1:1). Ro 20-1724 and PMSF are dissolved in ethyl alcohol. EGTA and EDTA are dissolved in 3N NaOH. Other drugs are dissolved in distilled water. The respective final concentrations of solvents are less 0.1% and did not significantly affect the activities of the PDE isozymes. All drug concentrations are presented in molarity.

In the present invention, quercetin-3-O-methylether (3-MQ) is isolated from Rhamnus nakaharai Hayata [27]. Quercetin-3,7,4′-O-trimethylether (ayanin) and quercetin-3,7,3′,4′-O-tetramethylether (QTME) are synthesized according to the method described by Gomm and Nierenstein [28], and quercetin-3,5,7,3′,4′-O-pentamethylether (QPME) is synthesized according to the method described by Kupchan and Bauerschmidt [29]. Quercetin-3,5,7,3′,4′-O-pentaacetate (QPA) and quercetin-3-O-methyl-5,7,3′,4′-O-tetraacetate (QMTA) are synthesized according to the method described by Ferte et al [30]. These quercetin derivatives are identified by spectral methods, including ultraviolet (UV), infrared (IR), mass spectroscopy (MS), and nuclear magnetic resonance (NMR) spectroscopic techniques. The purities of these compounds all exceeded 98% as determined by high-performance liquid chromatography (HPLC).

Hesperetin-5,7,3′-O-trimethylether (HTME) is synthesized according to the method described by Kupchan & Bauerschmidt [29]. First, 302.3 mg of hesperetin (1 mmol) are dissolved in 60 ml of acetone, and afterward 18 g of potassium carbonate and 6 ml dimethyl sulfate are added into the acetone. The mixed acetone is heated in silicone oil bath under 120□ for 18 hours, and the reacted acetone mixture is separated by silica gel column (where the mobile phase thereof is ethyl acetate:n-hexane=1:1) and crystallized by CH₂Cl₂ for obtaining 35 g yellow crystallized HTME (where the yield rate and mp thereof are 11.5% and 158-160□ respectively).

Hesperetin-7,3′-O-dimethylether (HDME) is synthesized according to the method described by Fumiss et al [31]. In the beginning, 100 mg of hesperetin are dissolved in the appropriate volume of dioxane and added the reacted diazomethane (yellow liquid) thereinto, and then this mixture is posited in the hood at the room temperature for about 18 hours. The reacted mixture is separated by silica gel column (where the mobile phase thereof is ethyl acetate:benzene:n-hexane=1:2:3) and fractionated by 15 ml per fraction. The fractions are analyzed by TLC for obtaining the distribution of HDME, and specific fractions containing HDME are collected and dried out to obtain raw HDME. The raw HDME is dissolved in a minimum volume of dichlomethane for purification. Then, large amounts of methanol are added in the dissolved raw HDME and took it in the ice bath for crystallization. After decreasing the pressure and filtering the mixture, white and crystallized HDME is obtained (where the yield rate and mp thereof are 32.7% and 132.5-134.4□ respectively).

Hesperetin-5,7,3′-O-triacetate (HTA) is synthesized according to the method described by Ferte et al [30]. Hesperetin (302.3 mg, 1 mmol) is orderly dissolved by 6 ml of pyridine and 6 ml of acetic anhydride in a beaker having a stir bar at the bottom thereof, and then the beaker is configured with the drying tube and the solution therein is stirred for reacting at the room temperature for 18 hours. The reacted solution is stopped by 5% w/v HCl and purified by methylene chloride. The raw HTA is separated by silica gel column (where the mobile phase thereof is EA:n-hexane=1:3) and crystallized by methanol for obtaining white and crystallized HTA (where the yield rate and mp thereof are 56% and 141-142□ respectively).

Separation of Cyclic Nucleotide PDE Isozymes

Under a protocol approved by the Animal Care and Use Committee of Taipei Medical University, five male guinea pigs (Hartley), weighing 500-600 g, are sacrificed. The lungs (15 g) or hearts (4 g) taken therefrom are chopped into small pieces and homogenized with a glass/teflon homogenizer (Glas-Col, Terre Haute, Ind., USA) in 10 volumes of cold medium (pH 6.5) containing 20 mM Bis-tris, 2 mM benzamidine, 2 mM EDTA, 50 mM sodium chloride, 0.1 mM phenylmethanesulfonyl fluoride (PMSF), and 1 mM dithiothreitol. At 4 , the homogenate is centrifuged at 170 g for 15 min, and the supernatant thereof is then re-centrifuged at 40,000 g for 30 min. The final supernatant fraction is filtered through the 0.22 μm filter and applied to a Q-Sepharose fast flow column (2.2×28 cm), which is pre-washed and pre-equilibrated in homogenization buffer. The column is washed with two bed volumes of homogenate buffer to remove unbound material, where the resin beads therein will bind with proteins, such as PDE proteins. Proteins bound to the Q-Sepharose beads are eluted with various concentrations (0.23, 0.34, 0.44, 0.69, and 1.00 M) of NaCl dissolved in homogenate buffer (for each concentration, 40-50 mL elution buffer are applied) at a flow rate of 30 ml/h. Fractions (3 ml each) are collected, and ethylene glycol (EG) is added thereto until a final concentration of 30% (v/v). And then the samples are frozen at −700. Under these conditions, the enzyme activity is stable for at least 3 months [32].

Assay I: Competitive Inhibition of Flavonoids on Cyclic Nucleotide PDE Activity

The activities of PDE4 in the homogenate are measured with a two-step procedure according to the method of Thompson and Appleman [33], using cAMP with [³H]-cAMP or cGMP with [³H]-cGMP as substrates. The PDE enzyme prepared (25 μl) with 10 μl inhibitor or the solvents therefor is incubated for 30 min at 37□ in a total assay buffer, where the final volume of the assay is amounted to 100 μl. In accordance with the features of the PDE isozymes, the assay buffer contains 50 mM Tris/HCl (pH 7.4), 3 mM MgCl₂, 1 mM dithiothreitol, and 0.05% BSA, and optionally contains 1 μM cAMP with 0.2 μCi [³H]-cAMP as a substrate alone or in the presence of 0.1 unit calmodulin with 10 μM CaCl₂, or 5 μM cGMP, and 1 μM cGMP with 0.2 μCi [³,H]-cGMP as another substrate alone or in the presence of 0.1 unit calmodulin with 10 μM CaCl₂. In assay of enzyme inhibition, the assay mixture contained with the inhibitors at various concentrations of flavonoids in the Table 1 or the PDE4 inhibitors as reference drugs, i.e. Rolipram and Ro 20-1724 [34].

The PDE enzyme and the inhibitors (or their solvent) therefor are mixed and incubated on ice for 30 min previously, and then mixed with the assay buffer. The assay is initiated by transferring the mixture to a water bath at 37□. Following the other 30 min incubation, the reaction is stopped by transferring the reaction vessel to a bath of boiling water for 3 min. After cooling on ice, 20 μl of the 1 mg/ml of Crotalus atrox venom is added to the reaction mixture, and the mixture is incubated at 37□ for 10 min. The uncatalyzed substrates, such as cAMP, [³H]-cAMP, cGMP, or [³H]-cGMP are removed by the addition of 500 μl of a 1-in-1 Tris-HCl (40 mM) buffer suspension of Dowex resin (1×8-200) with incubation on ice for 30 min, since the bindings of the cyclic nucleotides and the resin. Each tube is then centrifuged for 2 min at 6000 rpm (3700 g), and 100 μl of the supernatant is removed and counted by a β-counter for calculating the enzyme (PDEs) activity. Less than 10% of the tritiated cyclic nucleotide ([³H]-cAMP or [³H]-cGMP) is hydrolyzed in this assay.

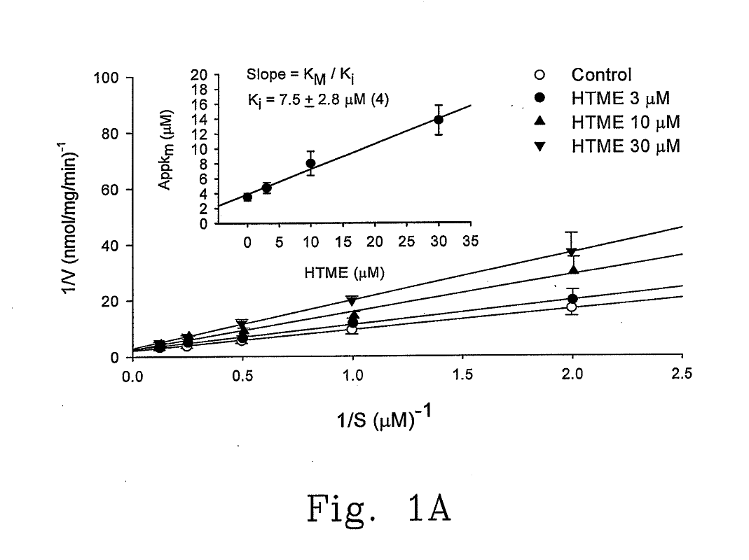

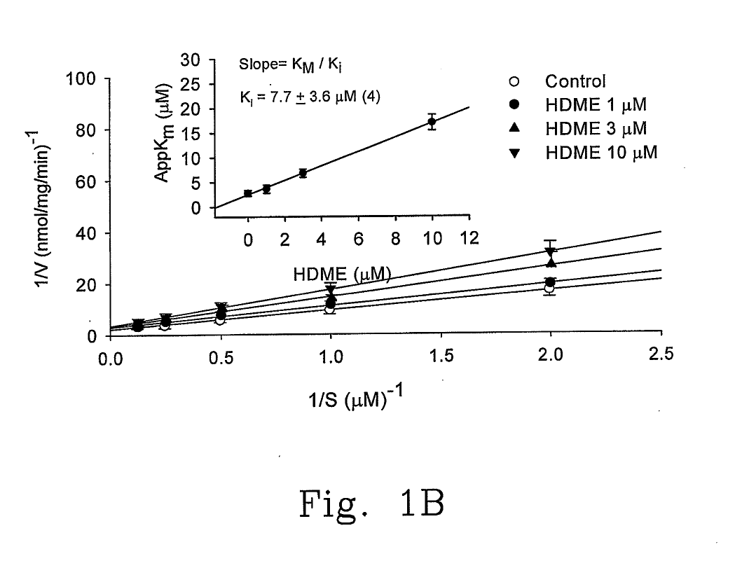

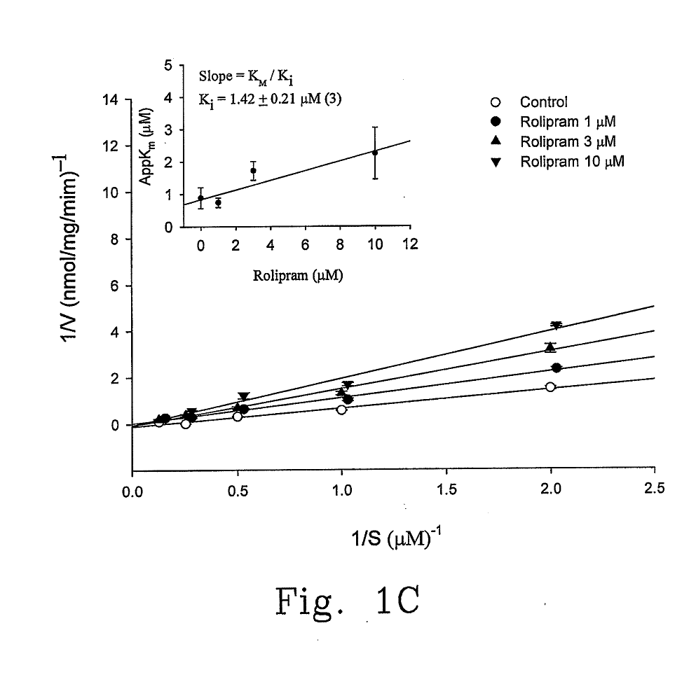

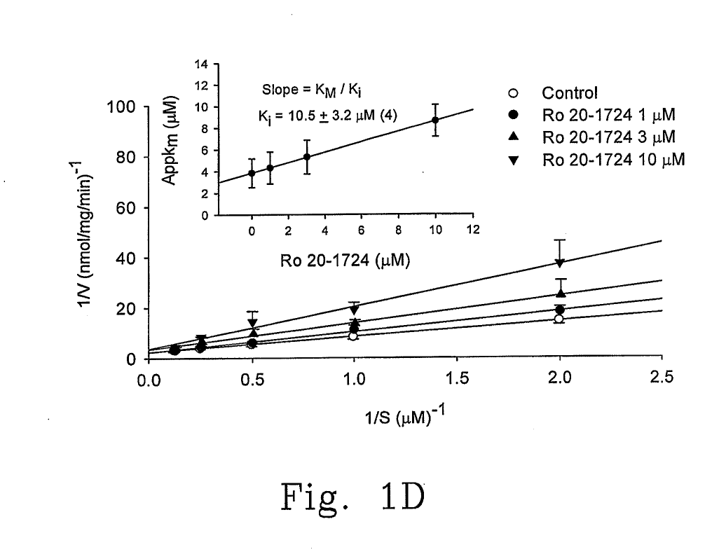

Please refer to FIGS. 1A to 1D, which show the enzyme activity of PDE4 for cAMP hydrolysis in the incubation of HTME, HDME, rolipram and Ro 20-1724 at various concentrations, respectively. While the activities of PDE4 (reaction velocity, shown as V) in the presence of various concentrations of hesperetin or rolipram and cAMP (substrate, shown as S) are analyzed in accordance to a double reciprocal plot, also called a Lineweaver-Burk plot [32]. The mode of action (such as competitive or non-competitive to PDE4) of hesperetin derivatives, rolipram or Ro 20-1724 is analyzed according to the plot. The dissociation constant of inhibitor binding (Kᵢ) value is determined from the equation of apparent Kₘ as a function of the inhibitor concentration (insert in A and B, respectively). The slope of the equation is equal to the value of K_{M}/Slope (where K_{M} is Michaelis constant). The amounts of the total proteins are calculated based on the analytical method reported by Bradford [35]. In FIGS. 1A to 1D, all enzyme activities are shown as nmole of substrate per mg of proteins per minute (nmole/mg/min) hydrolyzed. As shown in FIGS. 1A to 1D, hesperetin derivatives competitively inhibits the enzyme activity of PDE4.

Assay II: the Binding of Flavonoids to Particulate HARBS of Guinea Pig's Whole Brain

The binding experiments are carried on basis of the methods of Schneider et al [23] and Zhao et al [25] with a small modification. Under a protocol approved by the Animal Care and Use Committee of Taipei Medical University, five male guinea pigs (Hartley), weighing 500-600 g, are anesthetized. The whole brains taken therefrom are homogenized with in 10 volumes of cold medium (pH 6.5) containing 20 mM Bis-tris, 2 mM benzamidine, 2 mM EDTA, 50 mM sodium chloride, 0.1 mM phenylmethanesulfonyl fluoride (PMSF), and 1 mM dithiothreitol. At 4□, the homogenate is centrifuged at 170 g for 15 min for removing blood vessels and connective tissues, and the supernatant thereof is then re-centrifuged at 40,000 g for 30 min for separating the particulates from the cytosol of cells. The precipitated particulates are washed with fresh homogenate (4□) for several times, and the particulates are re-suspended to a 366 mg/ml of suspension (wet weight of brain per ml), where most of the particulates contained therein are cell membrane. It is found that there are 1.33 fmole [³H]-rolipram binding HARBS per mg of cell membrane after analysis by using Scatchard plots.

The respective binding abilities of the test drugs (flavonoids) to membrane HARBS are performed in a 25 μl of reaction solution, containing 10 μl of [³H]-rolipram, 10 μl of particulate suspension, and 5 μl of test drugs or selective PDE4 inhibitors for 60 min at 30° C., wherein the reaction buffer contained 50 mM Tris-HCl, 5 mM MgCl₂ (pH 7.5). The final concentration of [³H]-rolipram is 2 nM. Whereas, those of test drugs are ranged from 3-300 μM, and those of reference drugs (positive control), non-radioactive rolipram and Ro 20-1724, are ranged from 0.3-1000 nM and 1-10000 nM, respectively. After incubation, the reaction is stopped by transferring the reaction vessel to a bath of crashed ice. Then the reaction mixture is filtered through a punched glass fiber filter (Whatman GF/B) placed in a mini funnel, which is adopted to a 1.5 ml of Eppendorff's tube for collecting the filtrate in the mini centrifuge at 1000 rpm (100 g) for 10 seconds. By the same way (centrifugation), it is further washed with 0.3 ml reaction buffer each for three times. The Whatman GF/B filter is mixed with 2 ml of cocktail and counted by the β-scintillation counter (Backman, Fullerton, Calif., USA) for counting the radioactivity thereof.

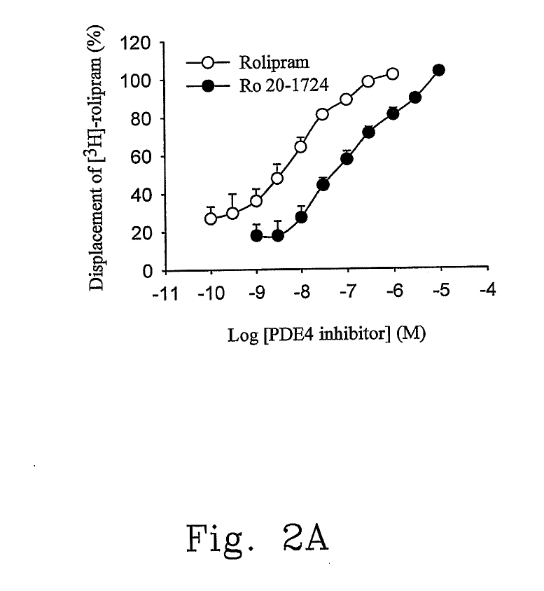

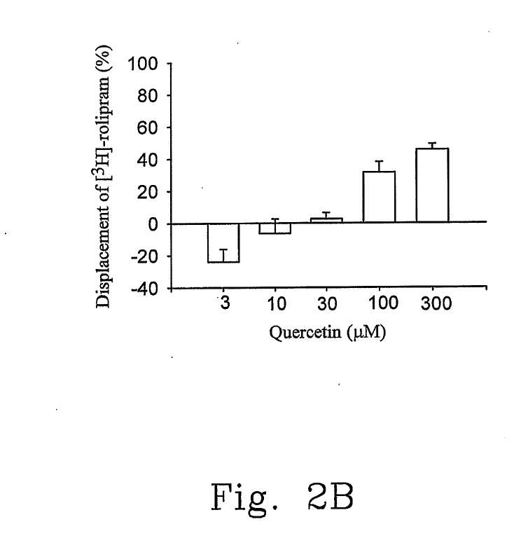

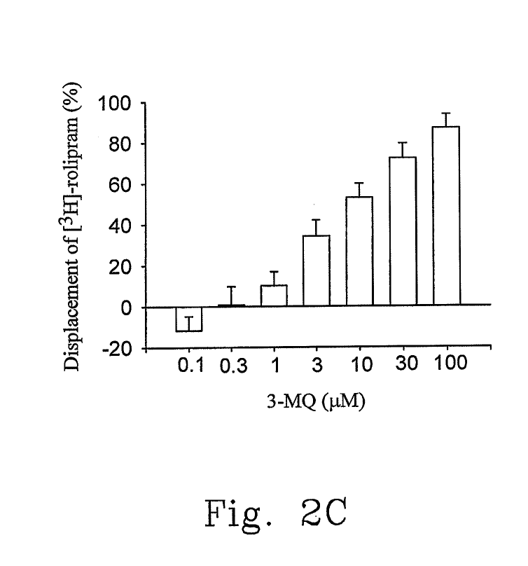

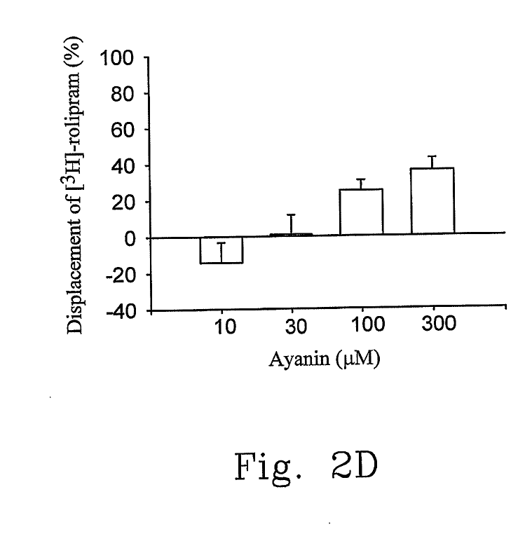

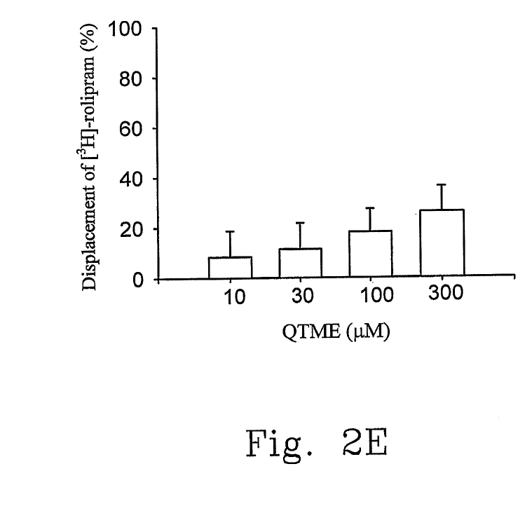

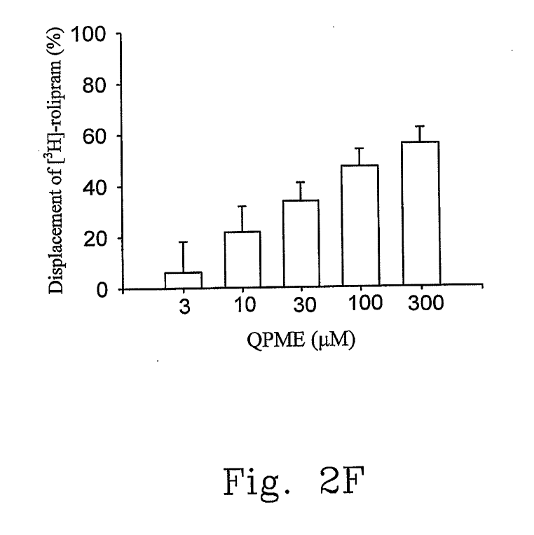

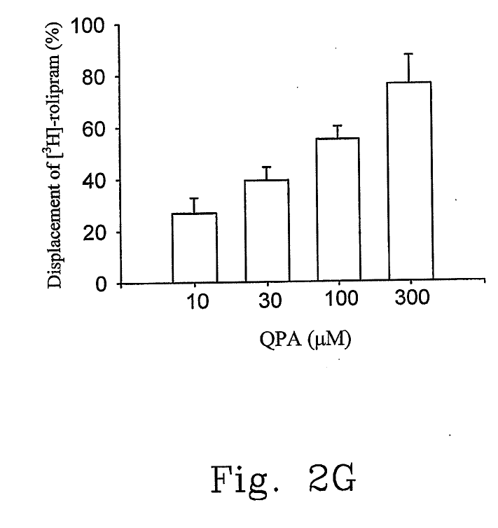

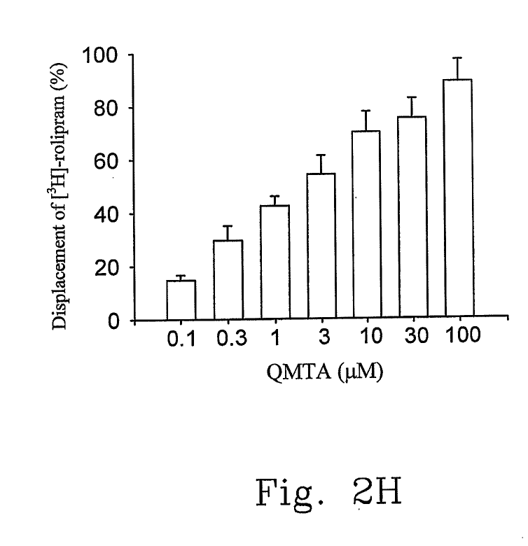

The median inhibition concentrations (IC₅₀s) of flavonoides for PDE4 activities are listed in the Table 2, where various derivatives of flavones, especially as quercetin derivatives and hesperetin derivatives, exhibited high PDE4_{H}/PDE4_{L} ratios which are apparently higher than those of reference drugs, rolipram and Ro 20-1724. Moreover, the concentration-dependent replacements of both rolipram and Ro 20-1724, quercetin, 3-MQ, ayanin, QTME, QPME, QPA and QMTA are respectively shown in FIGS. 2A to 2H [36]. As shown in FIGS. 2A to 2H, excellent EC₅₀ values of quercetin derivatives, obtained by the concentration of each quercetin derivatives that 50% of [³H]-rolipram bound on HARBSs are replaced by the quercetin derivatives, can be seen therein again.

Statistical Analysis

Concentrations of flavonoids at which 50% of maximum activity (EC₅₀ or IC₅₀ value) are produced are compared to each other. The EC₅₀ and IC₅₀ values of flavonoids and various reference drugs are calculated using non-linear regression analysis by the software SigmaPlot 10.0 (Sigma Chemical, St. Louis Mo., USA). All values are shown as the mean±S.E.M. Differences among values, which are equal or greater to three groups, are statistically calculated by one-way analysis of variance (ANOVA), and then determined by the least significant difference (LSD). The difference between two values, however, is determined by use of Student's unpaired t-test. Differences are considered statistically significant if the P-value is less than 0.05.

TABLE 1
Structures of flavonoids have the high PDE4_{H}/PDE4_{L} ratios

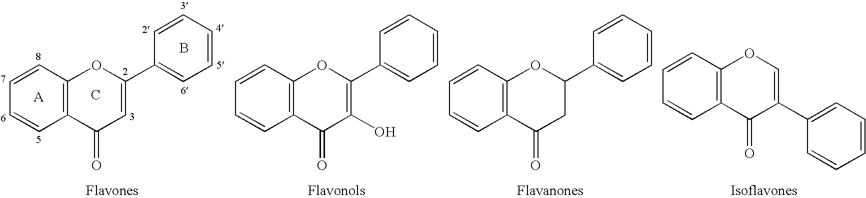

| | | Substitution | | | | | |
|---|---|---|---|---|---|---|---|
| Class | Name (Abbr.) | 3 | 5 | 7 | 3′ | 4′ | 5′ |
| Flavones | Luteolin-7-glucoside | | OH | O-glu | OH | OH | |
| | Diosmetin | | OH | OH | OH | OCH₃ | |
| Flavonols | Quercetin | OH | OH | OH | OH | OH | |
| | Qercetin-3-O-methylether (3-MQ) | OCH₃ | OH | OH | OH | OH | |
| | Quercetin-3,7,4′-O-trimethylether (Ayanin) | OCH₃ | OH | OCH₃ | OH | OCH₃ | |
| | Quercetin-3,7,3′,4′-O-tetramethylether (QTME) | OCH₃ | OH | OCH₃ | OCH₃ | OCH₃ | |
| | Quercetin-3,5,7,3′,4′-O-petamethylether (QPME) | OCH₃ | OCH₃ | OCH₃ | OCH₃ | OCH₃ | |
| | Quercetin-3,5,7,3′,4′-O-pentaacetate (QPA) | OCOCH₃ | OCOCH₃ | OCOCH₃ | OCOCH₃ | OCOCH₃ | |
| | Quercetin-3-O-methyl-5,7,3′,4′-O-tetraacetate (QMTA) | OCH₃ | OCOCH₃ | OCOCH₃ | OCOCH₃ | OCOCH₃ | |
| | Myricetin | OH | OH | OH | OH | OH | OH |
| Flavanones | Hesperetin | | OH | OH | OH | OCH₃ | |
| | Hesperetin-5,7,3′-O-trimethylether (HTME) | | OCH₃ | OCH₃ | OCH₃ | OCH₃ | |
| | Hesperetin-7,3′-O-dimethylether (HDME) | | OH | OCH₃ | OCH₃ | OCH₃ | |
| | Hesperetin-5,7,3′-O-triacetate (HTA) | | OCOCH₃ | OCOCH₃ | OCOCH₃ | OCH₃ | |
| Isoflavones | Genistein | | OH | OH | | OH | |
| | Biochanin A | | OH | OH | | OCH₃ | |
| | Prunetin | | OH | OCH₃ | | OH | |

Glu: glucose

TABLE 2
The IC₅₀ (μM) values of the mentioned flavonoids and the derivatives thereof on PDE 4 and their EC₅₀ values
(μM, unless indicated) for replacing high-affinity [³H]-rolipram binding

| | PDE4_{H} | | |
|---|---|---|---|
| Name (Abbr.) | (EC₅₀, μM, unless indicated) | PDE4_{L} (IC₅₀, μM) | PDE4_{H}/PDE4_{L} |
| Luteolin-7-glucoside | >300 | 43.0 ± 5.3 | >7 |
| Diosmetin | >300 | 20.2 ± 2.4 | >15 |
| Quercetin | >300 | 9.9 ± 2.5 | >30 |
| Qercetin-3-O-methylether (3-MQ) | 17.4 ± 4.0 | 28.5 ± 5.8 | 0.61 |
| Quercetin-3,7,4′-O-trimethylether (Ayanin) | >300 | 15.8 ± 4.4 | >19 |
| Quercetin-3,7,3′,4′-O-tetramethylether (QTME) | >300 | >100 | 3 |
| Quercetin-3,5,7,3′,4′-O-petamethylether (QPME) | 56.0 ± 22.7 | 5.1 ± 1.4 | 11 |
| Quercetin-3,5,7,3′,4′-O-pentaacetate (QPA) | 82.8 ± 22.3 | 18.5 ± 4.6 | 4.48 |
| Quercetin-3-O-methyl-5,7,3′,4′-O-tetraacetate (QMTA) | 3.9 ± 1.4 | 3.9 ± 0.6 | 1 |
| Myricetin | >300 | 39.8 ± 2.1 | >7.5 |
| Hesperetin | >300 | 28.2 ± 1.1 | >11 |
| Hesperetin-5,7,3′-O-trimethylether (HTME) | 171.4 ± 32.9 | 9.4 ± 2.9 | 18 |
| Hesperetin-7,3′-O-dimethylether (HDME) | 106.6 ± 39.5 | 3.0 ± 0.9 | 35.5 |
| Hesperetin-5,7,3′-O-triacetate (HTA) | >300 | 14.4 ± 1.8 | >21 |
| Genistein | 47.8 ± 15.9 | 9.5 ± 1.9 | 5.05 |
| Biochanin A | >300 | 8.5 ± 0.1 | >35 |
| Prunetin | >300 | 61.9 ± 17.3 | >4.8 |
| Reference Drug - Rolipram | 5.2 ± 1.9 nM | 2.3 ± 1.9 | 0.002 |
| Reference Drug - Ro 20-1724 | 87.0 ± 29.0 nM | 8.7 ± 1.9 | 0.01 |

EC₅₀: Effective concentration at which a half maximal HARBS is displaced
IC₅₀: Inhibitory concentration at which a half maximal PDE4 activity is inhibited, and the IC₅₀ values are reported to be similar to the effective concentration at a half maximal LARBS is displaced

While the invention has been described in terms of what are presently considered to be the most practical and preferred embodiments, it is to be understood that the invention need not be limited to the disclosed embodiment. On the contrary, it is intended to cover various modifications and similar arrangements included within the spirit and scope of the appended claims which are to be accorded with the broadest interpretation so as to encompass all such modifications and similar structures. Therefore, the above description and illustration should not be taken as limiting the scope of the present invention which is defined by the appended claims.

### REFERENCES

[1] Giembycz M A. Phosphodiesterase 4 inhibitors and the treatment of asthma. Drugs 2000; 59: 193-212.
[2] Torphy T J, Cieslinski L B. Characterization and selective inhibition of cyclic nucleotide phosphodiesterase isozymes in canine tracheal smooth muscle. Molecular Pharmacology 1990; 37: 206-214.
[3] De Boer J, Philpott A J, van Amsterdam R G, Shahid M, Zaagsma J, Nicholson C D. Human bronchial cyclic nucleotide phosphodiesterase isoenzymes: biochemical and pharmacological analysis using selective inhibitors. British Journal of Pharmacology 1992; 106: 1028-1034.
[4] Kapui Z, Schaeffer P, Mikus E G, Boronkay E, Gyürky J, Herbert J M, Pascal M. Experimental studies on guanosine 3′,5′-cyclic monophosphate levels and airway responsiveness of the novel phosphodiesterase type 5 inhibitor SR 265579 in guinea-pigs. Arzneimittel-Forschung 1999; 49: 685-693.
[5] Silver P J, Hamel L T, Perrone M H, Bentley R G, Bushover C R, Evans D B. Differential pharmacologic sensitivity of cyclic nucleotide phosphodiesterase isozymes isolated from cardiac muscle, arterial and airway smooth muscle. European Journal of Pharmacology 1988; 150: 85-94.
[6] Busse W W, Lemanske R F Jr. Asthma. New England Journal of Medicine 2001; 344: 350-362.
[7] Maddox L, Schwartz D A. The pathophysiology of asthma. Annual Review of Medicine 2002; 534: 477-498.
[8] Wills-Karp M. Immunologic basis of antigen-induced airway hyperresponsiveness. Annual Review of Immunology 1999; 17: 255-281.
[9] Kumar R K. Understanding airway wall remodeling in asthma: a basis for improvements in therapy? Pharmacology and Therapeutics 2001; 91: 93-104.
[10] Baumann J, von Bruchhausen F, Wurm G. Flavonoids and related compounds as inhibitors of arachidonic acid peroxidation. Prostaglandins 1980; 20: 627-639.
[11] Havsteen B. Flavonoids, a class of natural products of high pharmacological potency. Biochemical Pharmacology 1983; 32: 1141-1148.
[12] Wang H K, Xia Y, Yang Z Y, Natschke S L, Lee K H. Recent advances in the discovery and development of flavonoids and their analogues as antitumor and anti-HIV agents. Advances in Experimental Medicine & Biology 1998; 439: 191-225.
[13] Fotsis T, Pepper M S, Aktas E, Breit S, Rasku S, Adlercreutz H, Waehaelae K, Montesano R, Schweigerer L. Flavonoids, dietary-derived inhibitors of cell proliferation and in vitro angiogenesis. Cancer Research 1997; 57: 2916-2921.
[14] Kim H K, Cheon B S, Kim Y H, Kim S Y, Kim H P. Effects of Naturally Occurring Flavonoids on Nitric Oxide Production in the Macrophage Cell Line RAW264.7 and Their Structure-Activity Relationships. Biochemical Pharmacology 1999; 58: 759-765.
[15] Theoharides T C. Method of treating mast cell activation-induced diseases with a proteoglycan. U.S. Pat. No. 6,689,748 B1 2004.
[16] Akiyama T, Ishida J, Nakagawa S, Ogawara H, Watanabe S, Itoh N, Shibuya M, Fukami Y. Genistein a specific inhibitor of tyrosine-specific protein kinase. Journal of Biological Chemistry 1987; 262: 5592-5595.
[17] Nichols M R, Morimoto B H. Tyrosine kinase-independent inhibition of cyclic-AMP phosphodiesterase by Genistein and Tyrphostin 51. Archives of Biochemistry and Biophysics 1999; 366: 224-230.
[18] Nichols M R, Morimoto B H. Differential inhibition of multiple cAMP phosphodiesterase isozymes by isoflavones and tyrphostins. Molecular Pharmacology 2000; 57: 738-745.
[19] Underwood D C, Osborn R R, Novak L B, Matthews J K, Newsholme S J, Undem B J, Hand J M, Torphy T J. Inhibition of antigen-induced bronchoconstriction and eosinophil infiltration in the guinea pig by the cyclic AMP-specific phosphodiesterase inhibitor, rolipram. Journal of Pharmacology and Experimental Therapeutics 1993; 266: 306-313.
[20] Underwood D C, Kotzer C J, Bochnowicz S, Osborn R R, Luttmann M A, Hay D W, Torphy T J. Comparison of phosphodiesterase III, IV and dual III/IV inhibitors on bronchospasm and pulmonary eosinophil influx in guinea pigs. Journal of Pharmacology and Experimental Therapeutics 1994; 270: 250-259.
[21] Compton C H, Gubb J, Nieman R, Edelson J, Amit O, Bakst A, Ayres J G, Creemers J P, Schultze-Werninghaus G, Brambilla C, Barnes N C. Cilomilast, a selective phosphodiesterase-4 inhibitor for treatment of patients with chronic obstructive pulmonary disease: a randomised, dose-ranging study. Lancet 2001; 358: 265-270.
[22] Barnette M S, Bartus J O, Burman M, Christensen S B, Cieslinski L B, Esser K M, Prabhakar U S, Rush J A, Torphy T J. Association of the anti-inflammatory activity of phosphodiesterase 4 (PDE4) inhibitors with either inhibition of PDE4 catalytic activity or competition for [³H]rolipram binding. Biochemical Pharmacology 1996; 51: 949-956.
[23] Schneider H H, Schmiechen R, Brezinski M, Seidler J. Stereospecific binding of the antidepressant rolipram to brain protein structures. European Journal of Pharmacology 1986; 127: 105-115.
[24] Hatzelmann A, Schudt C. Anti-inflammatory and immunomodulatory potential of the novel PDE4 inhibitor roflumilast in vitro. Journal of Pharmacology and Experimental Therapeutics 2001; 297: 267-279.
[25] Zhao Y, Zhang H T, O'Donnell J M. Inhibitor binding to type 4 phosphodiesterase (PDE4) assessed using [³H]piclamilast and [³H]rolipram. Journal of Pharmacology and Experimental Therapeutics 2003; 305: 565-572.
[26] Draheim R, Egerland U, Rundfeldt C. Anti-inflammatory potential of the selective phosphodiesterase 4 inhibitor N-(3,5-dichloro-pyrid-4-yl)-[1-(4-fluorobenzyl)-5-hydroxy-indole-3-yl]-glyo xylic acid amide (AWD 12-281), in human cell preparations. Journal of Pharmacology and Experimental Therapeutics 2004; 308: 555-563.
[27] Lin C N, Lu C M, Lin H C, Ko F N, Teng C M. Novel antiplatelet naphthalene from Rhamnus nakaharai. Journal of Natural Products 1995; 58: 1934-1940.
[28] Gomm A S, Nierenstein M. The exhaustive O-methylation of quercetin. Journal of the American Chemical Society 1931; 53: 4408-4411.
[29] Kupchan S M, Bauerschmidt E. Cytotoxic flavonols from Baccharis sarothroides. Phytochemistry 1971; 10: 664-666.
[30] Ferte J, Kuhnel J M, Chapuis G, Rolland Y, Lewin G, Schwaller M A. Flavonoid-related modulators of multidrug resistance: synthesis, pharmacological activity, and structure-activity relationships. Journal of Medicinal Chemistry 1999; 42: 478-489.
[31] Furniss B S, Hannaford A J, Smith P W G, Tatchell A K. Preparation of diazomethane, In Vogel's Textbook of Practical Organic Chemistry. New York, Longman Scientific and Technical with John Wiley & Sons, 1989; [5th]: 432-433.
[32] Ko W C, Chen M C, Wang S H, Lai Y H, Chen J H, Lin C N. 3-O-methylquercetin more selectively inhibits phosphodiesterase subtype 3. Planta medica 2003; 69: 310-315.
[33] Thompson W J, Appleman M M. Multiple cyclic nucleotide phosphodiesterase activities from rat brain. Biochemistry 1971; 10: 311-316.
[34] Reeves M L, Leigh B K, England P J. The identification of a new cyclic nucleotide phosphodiesterase activity in human and guinea-pig cardiac ventricle. Implications for the mechanism of action of selective phosphodiesterase inhibitors. Biochemical Journal 1987; 241: 535-541.
[35] Bradford M M. A rapid and sensitive method for quantitation of microgram quantities of protein utilizing the principle of protein-dye binding. Analytical Biochemistry 1976; 72: 248-254.
[36] Chan Agnes L F, Huang H L, Chien H C, Chen C M, Lin C N, Ko W C. Inhibitory effects of quercetin derivatives on phosphodiesterase isozymes and high-affinity [³H]-rolipram binding in guinea pig tissues. Investigational New Drugs 2008; 26: 417-424.

What is claimed is:

1. A medical composition comprising:
a flavonoid as an active constituent having a binding affinity of high affinity rolipram binding sites (HARBS) of a phosphodiesterase 4 lower than a binding affinity of low affinity rolipram binding sites (LARBS) of the phosphodiesterase 4 and represented by formula (I): 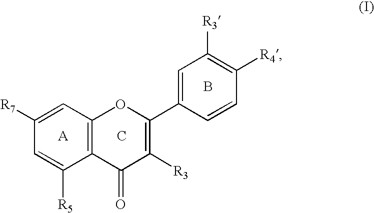
wherein each of R₃, R₅ and R₇ is one selected from the group consisting of hydrogen, hydroxyl, O-glu, O—R₈ and O-acyl-R₈,
each of R_{3′} and R_{4′} is one selected from the group consisting of hydrogen, hydroxyl, O—R₈ and O-acyl-R₈, and
R₈ is one selected from the group consisting of methyl, ethyl, n-propyl, i-propyl, n-butyl and i-butyl.

2. The medical composition according to claim 1, wherein the flavonoid is one selected from the group consisting of a luteolin, a luteolin-7-glucoside, a diosmetin, a quercetin and a myricetin.

3. The medical composition according to claim 1 having an effect of one of treating a chronic inflammation and being as a bronchodilator.

4. The medical composition according to claim 1 further comprising at least one of a medical excipient and a thinner.

5. A medical composition comprising:
a flavonoid as an active constituent having a binding affinity of high affinity rolipram binding sites (HARBS) of a phosphodiesterase 4 lower than a binding affinity of low affinity rolipram binding sites (LARBS) of the phosphodiesterase 4 and represented by formula (II): 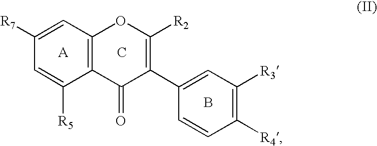
wherein each of R₂, R₅ and R₇ is one selected from the group consisting of hydrogen, hydroxyl, O—R₈ and O-acyl-R₈,
each of R_{3′} and R_{4′} is one selected from the group consisting of hydrogen, hydroxyl, O—R₈ and O-acyl-R₈, and
R₈ is one selected from a group consisting of methyl, ethyl, n-propyl, i-propyl, n-butyl, and i-butyl.

6. The medical composition according to claim 5, wherein the flavonoid is one selected from the group consisting of a genistein, a biochanin A, and a prunetin.

7. The medical composition according to claim 5 having an effect of one of treating a chronic inflammation and being as a bronchodilator.

8. The medical composition according to claim 5 further comprising one of a medical excipient or thinner.

9. A medical composition comprising:
a flavonoid as an active constituent having a binding affinity of high affinity rolipram binding sites (HARBS) of a phosphodiesterase 4 lower than a binding affinity of low affinity rolipram binding sites (LARBS) of the phosphodiesterase 4 and represented by formula (III): 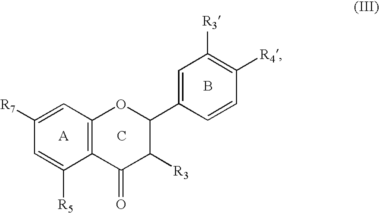
wherein each of R₃, R₅ and R₇ is one selected from the group consisting of hydrogen, hydroxyl O—R₈ and O-acyl-R₈,
each of R_{3′} and R_{4′} is one selected from the group consisting of hydrogen, hydroxyl, O—R₈ and O-acyl-R₈, and
R₈ is one selected from a group consisting of methyl, ethyl, n-propyl, i-propyl, n-butyl and i-butyl.

10. The medical composition according to claim 9, wherein the flavonoid is a hesperetin.

11. The medical composition according to claim 9 having an effect of one of treating a chronic inflammation and being as a bronchodilator.

12. The medical composition according to claim 9 further comprising at least one of a medical excipient and a thinner.
